(19) United States
(12) Patent Application Publication
(10) Pub. No.: US 2023/0414789 A1
(43) Pub. Date: Dec. 28, 2023

(54) SENSE INDICATOR COMPOSITION

(71) Applicant: CONTEMPORARY HEALTH SYSTEMS, LLC, Palm Beach, FL (US)

(72) Inventor: Ira Schneider, Palm Beach, FL (US)

(21) Appl. No.: 18/243,915

(22) Filed: Sep. 8, 2023

Related U.S. Application Data
(62) Division of application No. 17/375,591, filed on Jul. 14, 2021.
(60) Provisional application No. 63/052,162, filed on Jul. 15, 2020.

(51) Int. Cl. A61K 49/00

(52) U.S. Cl. CPC A61K 49/0004

(57) ABSTRACT

Compositions, kits, and methods for indicating a symptom of a disease. The composition may include one or more components having a taste or smell property as an indicator of a symptom of a disease. When applied to a user's tongue or mouth, detection or lack of detection of the taste or smell property may indicate a person has temporarily lost his/her sense of smell or taste as a result of a disease, particularly COVID-19 infection or exposure.

### CROSS REFERENCE

In accordance with 37 C.F.R. 1.76, a claim of priority is included in an Application Data Sheet filed concurrently herewith. Accordingly, the present invention is a Divisional application of U.S. application Ser. No. 17/375,591 entitled “SENSE INDICATOR COMPOSITION”, filed Jul. 14, 2021, which claims priority to U.S. Provisional Application No. 63/052,162, entitled “SENSE INDICATOR COMPOSITION”, filed Jul. 15, 2020. The contents of the above referenced application are incorporated herein by reference in their entirety.

### FIELD OF THE INVENTION

The present invention is related to compositions, kits, and methods for indicating a symptom of a disease; to compositions, kits, and methods of determining if an individual may have a diagnostic symptom associated with COVID-19 disease; and more particularly, to compositions, kits, and methods for deeming if a person has temporarily lost his/her sense of smell or taste as a result of COVID-19 infection or exposure.

### BACKGROUND OF THE INVENTION

The COVID-19 coronavirus pandemic has sparked world-wide fear and concern as the scientific and medical communities struggle to understand the virus and its disease pathology, infection cycle and spread. Since there is no currently known cure or effective medicinal treatments available, several non-pharmacological steps are being utilized in an attempt to prevent or slow the spread of the disease or minimize exposure to the virus. Such non-pharmacological steps include spraying of microbial disinfectants on widely used or public surfaces or areas, use of ultraviolet lights, social distancing, and wearing of masks when around others. While such systems may prevent the spread of disease, determining if a person is infected with the disease is critical.

Various testing mechanisms are known and are currently being utilized. For example, to determine if a person is infected with the virus, molecular tests which detect genetic material of the virus, or antigen based tests which detect virus protein are currently available. In addition to these tests, serological tests for detecting the presence of antibodies to the COVID-19 virus are currently being used to determine if an individual has been exposed to the COVID-19 virus. While such tests are currently being used, testing every person is not currently an option. Accordingly, testing is preferred to those individuals that show signs or symptoms of the disease or have been in close contact with someone they know has been diagnosed with the disease. While fever, cough, and shortness of breath are the key signs associated with COVID 19 infection, other symptoms have been reported by patients that may play a role in confirming patient infection.

As the disease has progressed since initial reports, a large subset of patients has been reporting anosmia, or loss of smell, as a symptom of COVID-19 infection or exposure. As more of the scientific community races towards developing a vaccine or pharmacological cure, mechanisms for helping determine if a person is suffering from COVID-19 so that individuals socially isolate themselves until healthy, and thus prevent further disease transmission, is needed.

### SUMMARY OF THE INVENTION

Embodied are devices, compositions, kits, and methods for use by individuals suspected of being infected with COVID-19. The devices, compositions, kits, and methods are used for indicating if a person has temporarily lost his/her sense of smell or taste as a result of COVID-19. The devices, compositions, kits, and methods for use by individuals suspected of being infected or exposed with COVID-19 may comprise taste or aroma properties to test sensory perceptions of taste or smell.

Accordingly, it is an objective of the invention to provide compositions having taste or aroma properties to test sensory perceptions of taste or smell.

It is a further objective of the invention to provide compositions having taste or aroma properties to test sensory perceptions of taste or smell as an indication of temporary loss of an individual's sense of smell or taste.

It is yet another objective of the invention to provide compositions having taste or aroma properties to test sensory perceptions of taste or smell as an indication of temporary loss of an individual's sense of smell or taste as a result of exposure to the COVID-19 virus.

It is a still further objective of the invention to provide compositions having taste or aroma properties to test sensory perceptions of taste or smell as an indication of COVID-19 viral infection or exposure.

It is an objective of the invention to provide multiple compositions in a kit, each composition having taste or aroma properties to test sensory perceptions of taste or smell.

It is a further objective of the invention to provide multiple compositions in a kit, each composition having taste or aroma properties to test sensory perceptions of taste or smell as an indication of temporary loss of an individual's sense of smell or taste.

It is yet another objective of the invention to provide multiple compositions in a kit, each composition having taste or aroma properties to test sensory perceptions of taste or smell as an indication of temporary loss of an individual's sense of smell or taste as a result of exposure to the COVID-19 virus.

It is a still further objective of the invention to provide multiple compositions in a kit, each composition having taste or aroma properties to test sensory perceptions of taste or smell as an indication of COVID-19 infection or exposure.

It is an objective of the invention to teach methods of using one or more compositions having taste or aroma properties to test sensory perceptions of taste or smell.

It is a further objective of the invention to teach methods of using one or more compositions having taste or aroma properties to test sensory perceptions of taste or smell as an indicator of temporary loss of an individual's sense of smell or taste.

It is yet another objective of the invention to teach methods of using one or more compositions having taste or aroma properties to test sensory perceptions of taste or smell as an indicator of temporary loss of an individual's sense of smell or taste as a result of exposure to the COVID-19 virus.

It is a still further objective of the invention to teach methods of using one or more compositions having taste or aroma properties to test sensory perceptions of taste or smell as an indicator of COVID-19 infection or exposure.

It is a further objective of the invention to provide compositions which can be safely positioned close to a user's nose and applied directly to a user's tongue as an indicator of temporary loss of an individual's sense of smell or taste as a result of exposure to the COVID-19 virus.

It is yet another objective of the invention to provide compositions which can be safely positioned close to a user's nose and applied directly to a user's tongue as an indicator of temporary loss of an individual's sense of smell or taste as an indicator of COVID-19 infection or exposure.

It is a still further objective of the invention to teach methods of applying compositions close to a user's nose and/or directly to a user's tongue to indicate temporary loss of an individual's sense of smell or taste as a result of exposure to the COVID-19 virus.

It is a still further objective of the invention to teach methods of applying compositions close to a user's nose and directly to a user's tongue to indicate temporary loss of an individual's sense of smell or taste as an indicator of COVID-19 infection or exposure.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1A illustrates a single embodiment of the sense indicator composition;

FIG. 1B illustrates the sense indicator in the form of a liquid and having a brush or sponge-type applicator;

FIG. 2 illustrates the sense indicator composition within a closed delivery unit;

FIG. 3 illustrates a kit including multiple sense indicator compositions, each having a different flavor or smell profile;

FIG. 4 illustrates the sense indicator composition applied to a person's nose; and

FIG. 5 illustrates the sense indicator composition applied to an individual's tongue.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

In certain embodiments of the invention, a plurality of ingredients is combined to form a unique composition, referred to generally as a sense indicator composition 10. The sense indicator composition 10 may comprise taste, aroma, or taste and aroma properties to test sensory perceptions of taste, smell, or taste and smell. The sense indicator composition 10 may be used as an indicator of having the virus responsible for COVID-19. Accordingly, administering the sense indicator composition 10 to an individual who is suspected of having COVID-19 may provide an indication or a non-indication of being positive for a symptom related to the COVID-19 infection.

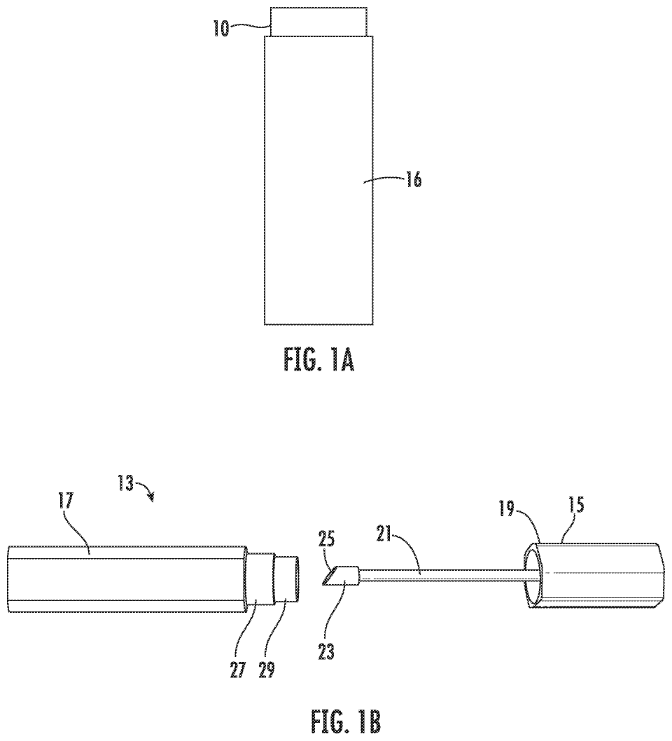

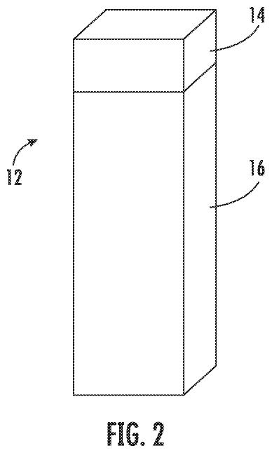

Referring to FIGS. 1A and 2, the sense indicator composition 10 may be housed in a delivery unit 12 having an upper portion 14 and a lower portion 16. Preferably, the upper portion 14 is removably attached to the lower portion 16. In this embodiment, a user simply removes the upper portion 14 from the lower portion 16, thus exposing the sense indicator composition 10, shown as a solid form, similar to a deodorant stick or lipstick. The delivery unit 12 may be configured with an actuating mechanism, such as a rotatable dial, that causes the sense indicator composition 10 to move up and out of, or down and into the lower portion 16.

The sense indicator composition 10 may be provided in alternative forms. For example, instead of a solid form, the sense indicator composition 10 may be made or provided in a liquid form, preferably as a viscous material. In a liquid form, the sense indicator composition 10 may be delivered using a different delivery unit 13. Referring to FIG. 1B, the delivery unit 12 is modified to allow for holding and dispensing of the liquid form of the sense indicator composition 10. The delivery unit 12 has an upper portion 15 and a lower portion 17. Preferably, the upper portion 15 is removably attached to the lower portion 17. The upper portion 15 includes a cap 19 and an applicator 21, illustrated herein as an elongated, generally rod-shaped body. The applicator 21 includes a tip 23, which may be made of a material, such as a foam like material, to suck up and hold the liquid form, of the sense indicator composition 10 stored within the lower portion 17. The applicator tip 23 may be rounding in shape and may include a tapered or angled surface 25. Extending from the lower portion 17 is a cylindrically shaped body 27 having external treading 29. The lower portion external treading 29 is sized and shaped to engage with corresponding internal threading (not shown) within the upper portion cap 19, thus providing a locking/unlocking mechanism to lock/unlock the upper portion 15 and the lower portion 17.

The sense indicator composition 10 may comprise of a single taste property to test sensory perceptions of taste. The sense indicator composition 10 may comprise of a plurality of taste properties to test sensory perceptions of taste. The sense indicator composition 10 may comprise of a single smell property to test sensory perceptions of smell. The sense indicator composition 10 may comprise of a plurality of taste properties to test sensory perceptions of taste. The sense indicator composition 10 may comprise of a single taste and smell property to test sensory perceptions of taste and smell. The sense indicator composition 10 may comprise of a plurality of taste and smell properties to test sensory perceptions of taste and smell.

In an illustrative embodiment, the sense indicator composition 10 may comprise an active ingredient having a taste and smell property as an indicator of a symptom of a disease; one or more non-active ingredients, wherein the active ingredient includes a sufficient concentration of an agent, such as a flavoring agent, having a taste and odor profile having taste and odor properties sufficient to test sensory perceptions of taste and smell of the user, wherein loss of recognition of taste or smell properties is an indicator of temporary loss of a user's sense of smell or taste as a result of exposure to COVID-19 virus.

TABLE 1
Sense indicator composition: Example 1

| Ingredient | Concentration |
|---|---|
| Active ingredients: Sense indicator | 3% or less |
| Inactive ingredients: Non-sense indicator | 97-99.9% |

TABLE 2
Sense indicator composition: Example 2

| Ingredient | Concentration |
|---|---|
| Flavoring agent | 3% or less |
| skin-conditioning agent: emollient, viscosity | 97-99.9% |
| increasing agent-nonaqueous, emollient, skin | |
| conditioning, and viscosity controlling; film | |
| former; occlusive; masking agent, and | |
| solvent; sweetener | |

TABLE 3
Sense indicator composition: Example 3

| Ingredient | Function | Concentration |
|---|---|---|
| Flavoring agent | taste and/or odor properties | 3% or less |
| hydrogenated | skin-conditioning agent- | 80-90% |
| polyisobutene | emollient, viscosity | |
| controlling | increasing agent- | |
| | nonaqueous, emollient, skin | |
| | conditioning, and viscosity | |
| ethylene/propylene/ | viscosity increasing agent- | 3-10% |
| styrene copolymer | nonaqueous | |
| butylene/ethylene/ | film former and film forming | |
| styrene copolymer: | | |
| cocos nucifera | skin-conditioning agent- | 5% or less |
| (coconut) oil: | miscellaneous; | |
| | skin-conditioning agent- | |
| | occlusive, emollient, | |
| | masking, skin conditioning, | |
| | and solvent | |
| stevioside | Sweetener | 5% or less |
| (steviol glycoside) | | |

TABLE 4
Sense indicator composition: Example 4

| Ingredient | Function | Concentration |
|---|---|---|
| Watermelon | taste and/or odor | 3% or less |
| Flavoring agent: | properties of | |
| | watermelon | |
| hydrogenated | skin-conditioning | 80-90% |
| polyisobutene | agent-emollient, | |
| controlling | viscosity increasing | |
| | agent-nonaqueous, | |
| | emollient, skin | |
| | conditioning, and | |
| | viscosity | |
| ethylene/propylene/ | viscosity increasing | 3-10% |
| styrene copolymer | agent-nonaqueous | |
| butylene/ethylene/ | film former and film | |
| styrene copolymer: | forming | |
| cocos nucifera | skin-conditioning | 5% or less |
| (coconut) oil: | agent-miscellaneous; | |
| | skin-conditioning | |
| | agent-occlusive, | |
| | emollient, masking, | |
| | skin conditioning, | |
| | and solvent | |
| stevioside | Sweetener | 5% or less |
| (steviol glycoside) | | |

TABLE 5
Sense indicator composition: Example 5

| Ingredient | Function | Concentration |
|---|---|---|
| Vanilla Flavoring | taste and/or odor | 3% or less |
| agent: | properties of vanilla | |
| hydrogenated | skin-conditioning agent- | 80-90% |
| polyisobutene | emollient, viscosity increasing | |
| controlling | agent-nonaqueous, emollient, | |
| | skin conditioning, and | |
| | viscosity | |
| ethylene/propylene/ | viscosity increasing agent- | 3-10% |
| styrene copolymer | nonaqueous | |
| butylene/ethylene/ | film former and film forming | |
| styrene copolymer: | | |
| cocos nucifera | skin-conditioning agent- | 5% or less |
| (coconut) oil: | miscellaneous; | |
| | skin-conditioning agent- | |
| | occlusive, emollient, masking, | |
| | skin conditioning, and solvent | |
| stevioside | Sweetener | 5% or less |
| (steviol glycoside) | | |

TABLE 6
Sense indicator composition: Example 6

| Ingredient | Function | Concentration |
|---|---|---|
| Mint Flavoring | taste and/or odor properties | 3% or less |
| agent: | of mint | |
| hydrogenated | skin-conditioning agent- | 80-90% |
| polyisobutene | emollient, viscosity | |
| controlling | increasing agent- | |
| | nonaqueous, emollient, skin | |
| | conditioning, and viscosity | |
| ethylene/propylene/ | viscosity increasing agent- | 3-10% |
| styrene copolymer | nonaqueous | |
| butylene/ethylene/ | film former and film forming | |
| styrene copolymer: | | |
| cocos nucifera | skin-conditioning agent- | 5% or less |
| (coconut) oil: | miscellaneous; | |
| | skin-conditioning agent- | |
| | occlusive, emollient, | |
| | masking, skin conditioning, | |
| | and solvent | |
| stevioside | Sweetener | 5% or less |
| (steviol glycoside) | | |

TABLE 7
Sense indicator composition: Example 7

| Ingredient | Function | Concentration |
|---|---|---|
| Coffee Flavoring | taste and/or odor | 3% or less |
| agent: | properties of coffee | |
| hydrogenated | skin-conditioning | 80-90% |
| polyisobutene | agent-emollient, | |
| controlling | viscosity increasing | |
| | agent-nonaqueous, | |
| | emollient, skin | |
| | conditioning, and | |
| | viscosity | |
| ethylene/propylene/ | viscosity increasing | 3-10% |
| styrene copolymer | agent-nonaqueous | |
| butylene/ethylene/ | film former and film | |
| styrene copolymer: | forming | |
| cocos nucifera | skin-conditioning | 5% or less |
| (coconut) oil: | agent-miscellaneous; | |
| | skin-conditioning | |
| | agent-occlusive, | |
| | emollient, masking, | |
| | skin conditioning, | |
| | and solvent | |
| stevioside | Sweetener | 5% or less |
| (steviol glycoside) | | |

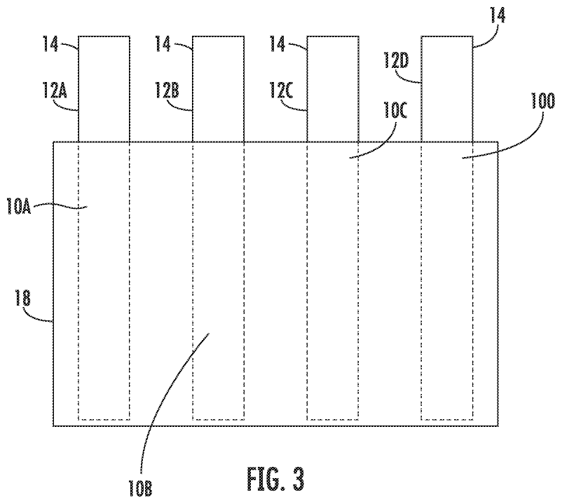

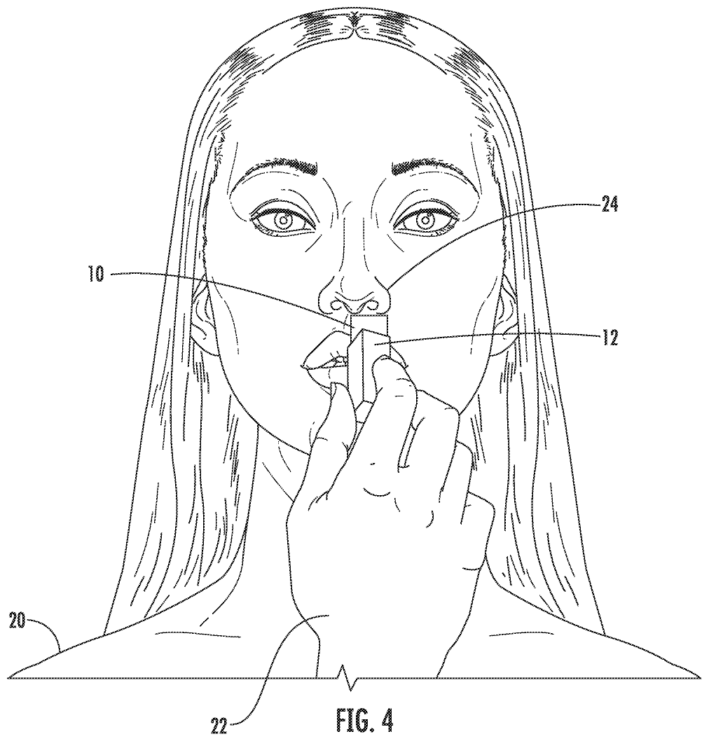

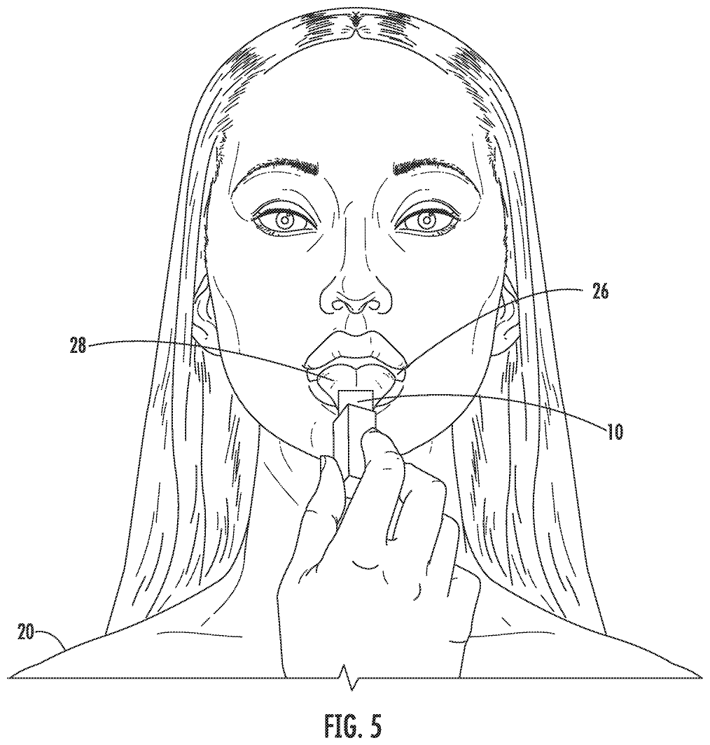

The sense indicator composition 10 may be provided as a single use composition. Alternatively, the sense indicator composition 10 may be provided as a kit with multiple compositions. Referring to FIG. 3, multiple sense indicator composition applicators 12 (referred to individually as 12A, 12B, 12C, or 12D) are shown in a container 18. The container 18 houses four sense indicator compositions 10, sense indicator composition 10A with watermelon taste and smell properties, sense indicator composition 10B with vanilla taste and smell properties, sense indicator composition 10C with mint taste and smell properties, and sense indicator composition 10D with coffee taste and smell properties.

The sense indicator composition 10 is configured to provide taste or smell proprieties as an indicator of temporary loss of an individual's sense of smell or taste as a result of exposure to COVID-19 virus. Accordingly, in use, a user 20 places a sense indicator composition 10, via applicator 12, in his/her hand 22 and positions the sense indicator composition 10 just below his/her nose 24. Alternatively, the user may use the delivery device 12 to position the sense indicator composition 10 at a sufficient distance from the nose so a user can smell the odor associated with the flavor property/properties. If the individual can smell the particular flavor property/properties, he/she may not be suffering from the loss of smell as a symptom of COVID-19 infection. If the user is unable to smell the particular flavor property/properties, i.e. has lost the ability to smell the flavor profile, such inability to do so may indicate a symptom of being infected with COVID-19.

Alternatively, or in addition to application to a user's nose, the user 20 may apply the sense indicator composition 10 to the user's mouth 26, applying the sense indicator composition 10 directly to the user's tongue 28. If the individual can taste the particular flavor property/properties, he/she may not be suffering from the loss of taste as a symptom of COVID-19 infection. If the user is unable to taste the particular flavor property/properties, i.e. has lost the ability to detect the flavor profile, such inability to do so may indicate a symptom of being infected with COVID-19. Accordingly, the sense indicator composition 10 is preferably made of ingredients that are safe for human consumption.

While the sense indicator composition 10 has been described to assume a solid form, other forms may be suitable, such as a spray, a roll-on or roll-on ball, a cream, a semi-solid, gel, foam, or other forms known to one of skill in the art. In addition, while the sense indicator composition 10 has been embodied with particular flavor agents or other composition ingredients, such as occlusives, emollients, masking agents, skin conditioning agents, and solvents, etc., other specific chemicals with the same functions known to one of skill in the art may be used or substituted.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of determining loss of taste and smell in an individual comprising:
providing a first diagnostic sense indicator device comprising a composition comprising one or more active ingredients and one or more inactive ingredients, said one or more active ingredients having a predetermined taste and smell characteristic when applied to a user's nose and mouth, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said first diagnostic sense indicator device to said user's nose;
determining said smell characteristic associated with said first diagnostic sense indicator device by obtaining a response to exposure of said first diagnostic sense indicator device from said user, wherein if said user is unable to identify the predetermined smell characteristic, said user may be suffering from the loss of smell;
exposing said first diagnostic sense indicator device to said user's mouth; and
determining said taste characteristic associated with said first diagnostic sense indicator device by obtaining a response to exposure of said first diagnostic sense indicator device from said user, wherein if said user is unable to identify said predetermined taste characteristic, said user may be suffering from a loss of taste;
providing a second diagnostic sense indicator device comprising a composition comprising one or more active ingredients and one or more inactive ingredients, said one or more active ingredients having a predetermined taste and smell characteristic when applied to said user's nose and mouth, said second diagnostic sense indicator device composition taste and smell characteristic being a different taste and smell characteristic associated with said first diagnostic sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said second diagnostic sense indicator device to said user's nose;
determining said smell characteristic associated with said second diagnostic sense indicator device by obtaining a response to exposure of said second diagnostic sense indicator device from said user, wherein if said user is unable to identify said predetermined smell characteristic, said user may be suffering from a loss of smell.
exposing said second diagnostic sense indicator device to said user's mouth; and
determining said taste characteristic associated with said second diagnostic sense indicator device by obtaining a response to exposure of said second diagnostic sense indicator device from said user, wherein if said user is unable to identify said predetermined taste characteristic, said user may be suffering from a loss of taste;
providing a third diagnostic sense indicator device comprising a composition comprising one or more active ingredients and one or more inactive ingredients, said one or more active ingredients having a predetermined taste and smell characteristic when applied to said user's nose and mouth, said third diagnostic sense indicator device composition taste and smell characteristic being a different taste and smell characteristic associated with said first or second diagnostic sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said third diagnostic sense indicator device to said user's nose;
determining said smell characteristic associated with said third diagnostic sense indicator device by obtaining a response to exposure of said third diagnostic sense indicator device from said user, wherein if said user is unable to identify said predetermined smell characteristic, said user may be suffering from a loss of smell;
exposing said third diagnostic sense indicator device to said user's mouth; and
determining said taste characteristic associated with said third diagnostic sense indicator device by obtaining a response to exposure of said third diagnostic sense indicator device from said user, wherein if said user is unable to identify said predetermined taste characteristic, said user may be suffering from a loss of taste;
providing a fourth diagnostic sense indicator device comprising a composition comprising one or more active ingredients and one or more inactive ingredients, said one or more active ingredients having a predetermined taste and smell characteristic when applied to said user's nose and mouth, said fourth diagnostic sense indicator device composition taste and smell characteristic being a different taste and smell characteristic associated with said first, second, or third sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said fourth diagnostic sense indicator device to said user's nose;
determining said smell characteristic associated with said fourth diagnostic sense indicator device by obtaining a response to exposure of said fourth diagnostic sense indicator device from said user, wherein if said user is unable to identify said predetermined smell characteristic, said user may be suffering from a loss of smell;
exposing said fourth diagnostic sense indicator device to said user's mouth; and
determining said taste characteristic associated with said fourth diagnostic sense indicator device by obtaining a response to exposure of said fourth diagnostic sense indicator device from said user, wherein if said user is unable to identify said predetermined taste characteristic, said user may be suffering from a loss of taste.

2. The method of determining loss of taste and smell in an individual according to claim 1, further including:
providing a second diagnostic sense indicator device comprising a composition comprising one or more active ingredients and one or more inactive ingredients, said one or more active ingredients having a predetermined taste and smell characteristic when applied to said user's nose and mouth, said second diagnostic sense indicator device composition taste and smell characteristic being a different taste and smell characteristic associated with said first diagnostic sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said second diagnostic sense indicator device to said user's nose;
determining said smell characteristic associated with said second diagnostic sense indicator device by obtaining a response to exposure of said second diagnostic sense indicator device from said user, wherein if said user is unable to identify said predetermined smell characteristic, said user may be suffering from a loss of smell.
exposing said second diagnostic sense indicator device to said user's mouth; and
determining said taste characteristic associated with said second diagnostic sense indicator device by obtaining a response to exposure of said second diagnostic sense indicator device from said user, wherein if said user is unable to identify said predetermined taste characteristic, said user may be suffering from a loss of taste.

3. The method of determining loss of taste and smell in an individual according to claim 2, further including:
providing a third diagnostic sense indicator device comprising a composition comprising one or more active ingredients and one or more inactive ingredients, said one or more active ingredients having a predetermined taste and smell characteristic when applied to said user's nose and mouth, said third diagnostic sense indicator device composition taste and smell characteristic being a different taste and smell characteristic associated with said first or second diagnostic sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said third diagnostic sense indicator device to said user's nose;
determining said smell characteristic associated with said third diagnostic sense indicator device by obtaining a response to exposure of said third diagnostic sense indicator device from said user, wherein if said user is unable to identify said predetermined smell characteristic, said user may be suffering from a loss of smell;
exposing said third diagnostic sense indicator device to said user's mouth; and
determining said taste characteristic associated with said third diagnostic sense indicator device by obtaining a response to exposure of said third diagnostic sense indicator device from said user, wherein if said user is unable to identify said predetermined taste characteristic, said user may be suffering from a loss of taste.

4. The method of determining loss of taste and smell in an individual according to claim 3, further including:
providing a fourth diagnostic sense indicator device comprising a composition comprising one or more active ingredients and one or more inactive ingredients, said one or more active ingredients having a predetermined taste and smell characteristic when applied to said user's nose and mouth, said fourth diagnostic sense indicator device composition taste and smell characteristic being a different taste and smell characteristic associated with said first, second, or third sense indicator device composition, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
exposing said fourth diagnostic sense indicator device to said user's nose;
determining said smell characteristic associated with said fourth diagnostic sense indicator device by obtaining a response to exposure of said fourth diagnostic sense indicator device from said user, wherein if said user is unable to identify said predetermined smell characteristic, said user may be suffering from a loss of smell;
exposing said fourth diagnostic sense indicator device to said user's mouth; and
determining said taste characteristic associated with said fourth diagnostic sense indicator device by obtaining a response to exposure of said fourth diagnostic sense indicator device from said user, wherein if said user is unable to identify said predetermined taste characteristic, said user may be suffering from a loss of taste.

5. The method of determining loss of taste and smell in an individual according to claim 1, wherein,
one of said first, second, third, and fourth diagnostic sense indicator device taste and smell characteristics represent bitterness,
one of said first, second, third, and fourth diagnostic sense indicator device taste and smell characteristics represent sourness;
one of said first, second, third, and fourth diagnostic sense indicator device taste and smell characteristics represent sweetness; and
one of said first, second, third, and fourth diagnostic sense indicator device taste and smell characteristics represent saltiness.

6. The method of determining loss of taste and smell in an individual according to claim 1, wherein, said first, second, third, and fourth diagnostic sense indicator device compositions are formulated as a liquid material or a solid material.

7. A method of using a diagnostic sense indicator device for determining loss of taste and smell in an individual comprising:
providing a first diagnostic sense indicator device comprising a composition comprising one or more active ingredients and one or more inactive ingredients, said one or more active ingredients having a predetermined taste and smell characteristic when applied to a user's nose and mouth, said taste and smell characteristic representing one type of the four recognized taste and smell types, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
providing a second diagnostic sense indicator device comprising a composition comprising one or more active ingredients and one or more inactive ingredients, said one or more active ingredients having a predetermined taste and smell characteristic when applied to said user's nose and mouth, said taste and smell characteristic representing a second type of said four recognized taste and smell types, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
providing a third diagnostic sense indicator device comprising a composition comprising one or more active ingredients and one or more inactive ingredients, said one or more active ingredients having a predetermined taste and smell characteristic when applied to said user's nose and mouth, said taste and smell characteristic representing a third type of said four recognized taste and smell types, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
providing a fourth diagnostic sense indicator device comprising a composition comprising one or more active ingredients and one or more inactive ingredients, said one or more active ingredients having a predetermined taste and smell characteristic when applied to said user's nose and mouth, said taste and smell characteristic representing a fourth type of said four recognized taste and smell types, said one or more non-active ingredients includes one or more of an emollient, a viscosity increasing agent, a viscosity controlling agent, a film former, an occlusive, a masking agent, a solvent, a sweetener, or combinations thereof, and an applicator configured to enclose said composition when not in use and deliver said composition to said user's nose or mouth when in use;
administering said first diagnostic sense indicator device to said user's nose;
evaluating said user by obtaining a response from said user to said smell characteristic associated with said first diagnostic sense indicator device, wherein if said user is unable to identify said predetermined smell characteristic, said user may be suffering from a loss of smell,
administering said first diagnostic sense indicator device to said user's mouth;
evaluating said user by obtaining a response from said user to said taste characteristic associated with said first diagnostic sense indicator device, wherein if said user is unable to identify said predetermined taste characteristic, said user may be suffering from a loss of taste;
administering said second diagnostic sense indicator device to said user's nose;
evaluating said user by obtaining a response from said user to said smell characteristic associated with said second diagnostic sense indicator device, wherein if said user is unable to identify said predetermined smell characteristic, said user may be suffering a loss of smell;
administering said second diagnostic sense indicator device to said user's mouth,
evaluating said user by obtaining a response from said user to said taste characteristic associated with said second diagnostic sense indicator device, wherein if said user is unable to identify said predetermined taste characteristic, said user may be suffering a loss of taste;
administering said third diagnostic sense indicator device to said user's nose;
evaluating said user by obtaining a response from said user to said smell characteristic associated with said third diagnostic sense indicator device, wherein if said user is unable to identify said predetermined smell characteristic, said user may be suffering a loss of smell;
administering said third diagnostic sense indicator device to said user's mouth;
evaluating said user by obtaining a response from said user to said taste characteristic associated with said third diagnostic sense indicator device, wherein if said user is unable to identify said predetermined taste characteristic, said user may be suffering a loss of taste;
administering said fourth diagnostic sense indicator device to said user's nose;
evaluating said user by obtaining a response from said user to said smell characteristic associated with said fourth diagnostic sense indicator device, wherein if said user is unable to identify said predetermined smell characteristic, said user may be suffering a loss of smell;
administering said fourth diagnostic sense indicator device to said user's mouth; and
evaluating said user by obtaining a response from said user to said taste characteristic associated with said fourth diagnostic sense indicator device, wherein if said user is unable to identify said predetermined smell characteristic, said user may be suffering a loss of taste.

8. The method of using a diagnostic sense indicator device for determining loss of taste and smell in an individual according to claim 7, wherein,
one of said first, second, third, and fourth diagnostic sense indicator device taste and smell characteristics incudes a smell and taste representing bitterness,
one of said first, second, third, and fourth diagnostic sense indicator device taste and smell characteristics incudes a smell and taste representing sourness;
one of said first, second, third, and fourth diagnostic sense indicator device taste and smell characteristics incudes a smell and taste representing sweetness; and
one of said first, second, third, and fourth diagnostic sense indicator device taste and smell characteristics incudes a smell and taste representing saltiness.

9. The method of using a diagnostic sense indicator device for determining loss of taste and smell in an individual according to claim 7, wherein said first, second, third, and fourth diagnostic sense indicator device compositions are formulated as a liquid material.

10. The method of using a diagnostic sense indicator device for determining loss of taste and smell in an individual according to claim 7, wherein said first, second, third, and fourth diagnostic sense indicator device compositions are formulated as a solid material.

11. The method of using a diagnostic sense indicator device for determining loss of taste and smell in an individual according to claim 7, wherein said first, second, third, or fourth diagnostic sense indicator devices are applied to said user's lip, tongue, or lip and tongue.

12. The method of using a diagnostic sense indicator device for determining loss of taste and smell in an individual according to claim 7, wherein, a determination of loss of taste or smell to any one of said first, second, third, or fourth diagnostic sense indicator devices is a diagnostic indicator of a symptom of a disease or medical condition.
